(19) United States
(12) Patent Application Publication
(10) Pub. No.: US 2010/0198012 A1
(43) Pub. Date: Aug. 5, 2010

(54) APPARATUS FOR DEPLOYING ENDOSCOPE

(75) Inventors: George Anthony Poole, Lymington (GB); Antony John Young, Lymington (GB)

Correspondence Address: Workman Nydegger;1000 Eagle Gate Tower, 60 East South Temple, Salt Lake City, UT, 84111, US

(73) Assignee: SCOPEGUARD LIMITED, Liphook, Hampshire (GB)

(21) Appl. No.: 12/594,564

(22) PCT Filed: Apr. 7, 2008
(86) PCT No.: PCT/GB2008/001204

§ 371 Date: Apr. 5, 2010

(30) Foreign Application Priority Data
Apr. 5, 2007 (GB) 0706783.8
Feb. 12, 2008 (GB) 0802583.5

(51) Int. Cl. A61B 1/00

(52) U.S. Cl. 600/115; 600/121; 600/114; 600/123

(57) ABSTRACT

Apparatus for inserting an instrument (1) in a flexible body cavity is disclosed. The apparatus includes a sheath (3) having an inner chamber (31) for accommodating the instrument (1) and an outer chamber (41) surrounding the inner chamber, thereby defining an enclosed space between the inner chamber and the outer chamber Fluid pressure is temporarily applied to thse enclosed space so as to expand the outer chamber to facilitate insertion of the instrument in the body cavity. In another embodiment the inner chamber is expandable to have a diameter equal to or greater than the diameter of the outer chamber, and wherein the outer chamber incorporates one or more lines of weakness for facilitating release and expansion of the inner chamber and withdrawal of the instrument from the inner chamber. In yet another embodiment the sheath includes at its distal end a convex lens for use by a camera in obtaining images of said body cavity. A flexible housing for receiving the instrument may be provided with a relatively rigid intermediate part between the housing and the sheath for guiding the instrument from said housing to said sheath.

### TECHNICAL FIELD

The present invention relates to apparatus for inserting an instrument in a body cavity, apparatus for applying an instrument to a body cavity, apparatus for inserting imaging means into a body cavity, and an endoscope end cap.

### BACKGROUND TO THE INVENTION

An endoscope is a device used to look inside a body cavity, such as a cavity of the human or animal body. When the body cavity is that of a human or animal body, the endoscope is inserted through a natural opening, such as the mouth during a bronchoscopy, the rectum for a sigmoidoscopy or into the bladder to perform a cystoscopy. A cystoscopy is performed with a cystoscope. Generally the scope transmits the image from the inside of the body cavity along its shaft to the operator or a video screen via a fibre optic bundle or a video chip.

For example, a cystoscopy procedure may include initially cleaning the uretheral meatus and applying a local anaesthetic jelly. The cystoscope is then inserted through the urethera into the bladder. Water or saline is injected through the cystoscope to fill the bladder. As the fluid fills the bladder, it stretches the bladder wall which enables the cystoscope operator to view the entire bladder wall.

The Applicant's European Patent No. 1315444 discloses a body cavity liner or sheath for deploying an endoscope. The subject-matter of that patent is fully incorporated herein by reference. In that patent an endoscope is located in a chamber. Further chambers are provided. These further chambers may be used as “working channels”. These working channels provide passage for medical instruments. The working channels may be formed longitudinally by the application of heat to the wall of the main chamber and/or the working channels.

Known sheath arrangements for deploying endoscopes suffer from various problems.

For example, access of the sheath/scope assembly into the body is impeded by small orifices in the body leading to long narrow tubes. This tends to cause the sheath material to fold or wrinkle at the entrance to the orifice which obstructs insertion of the sheath/scope assembly and is very painful for the patient.

A further problem exists during the removal of the endoscope from the sheath due to friction. By their very nature sheaths need to be as close a fit to the endoscope as possible, and may typically be 0.5 mm diameter larger. Consequently, when the endoscope is removed from the sheath wrinkling or folds of the sheath tend to occur. As there is minimal free tolerance, any folds would decrease the diameter of the sheath and restrict free withdrawal of the scope, to the point of lock up.

Various other problems associated with the known sheath/endoscope assemblies are discussed herein and various improvements to known sheath/endoscope assembly arrangements are described.

### BRIEF SUMMARY OF THE INVENTION

According to a first aspect of the present invention, there is provided apparatus for inserting an instrument in a body cavity, the apparatus including a sheath having an inner chamber for accommodating the instrument and an outer chamber surrounding the inner chamber, thereby defining an enclosed space between the inner chamber and the outer chamber; and means for temporarily applying fluid pressure to said enclosed space so as to expand the outer chamber to facilitate insertion of the instrument in the body cavity.

According to a second aspect of the present invention, there is provided apparatus for applying an instrument to a body cavity, the apparatus including a sheath having an inner chamber for accommodating the instrument and an outer chamber surrounding the inner chamber, wherein the inner chamber is expandable to have a diameter equal to or greater than the diameter of the outer chamber, and wherein the outer chamber incorporates one or more lines of weakness for facilitating release and expansion of the inner chamber and withdrawal of the instrument from the inner chamber. Alternatively the inner chamber could have the line(s) of weakness and the outer sheath be expandable, or both chambers have line(s) of weakness and be expandable.

The inner chamber may be folded/pleated/twisted to reduce the diameter of the chamber so that it fits within the outer chamber. Once the outer chamber is released through the failure mechanism, the scope can be easily removed from the inner chamber due to the diameter of the inner chamber then being appreciably larger than that of the endoscope.

According to a third aspect of the present invention, there is provided apparatus for inserting imaging means into a body cavity, the apparatus including a sheath for accommodating the imaging means, wherein said sheath includes at its proximal end a convex lens for use by said imaging means in obtaining images of said body cavity.

The imaging means may comprise a camera or image transmitting mechanism, such as a fibre optic cable.

According to a fourth aspect of the present invention, there is provided apparatus for inserting an instrument into a body cavity, the apparatus including:
a sheath for accommodating the instrument;
a flexible housing for receiving the instrument;
a relatively rigid intermediate part between said housing and said sheath for guiding the instrument from said housing to said sheath.

According to a fifth aspect of the present invention, there is provided apparatus for inserting an instrument into a body cavity, the apparatus including a sheath for accommodating the instrument, wherein the sheath includes phosphorycholine. For example, the sheath may be coated with phosphorycholine-containing polymers. Such a coating may advantageously reduce friction between the sheath and the body cavity and/or the sheath and the instrument. Additionally or alternatively the coating may reduce adhesion of bacteria in the body cavity to the sheath, thereby reducing potential transportation of bacteria along the body cavity as the sheath moves along the body cavity. Suitable phosphorycholine materials are available from Vertellus of Basingstoke, United Kingdom. Suitable materials are described in EP-A-0 593 561.

According to a sixth aspect of the present invention, there is provided an endoscope (or other medical viewing device) end cap including a lens and a formation for mechanically fitting the end cap to the endoscope such that the lens is aligned with a part of the endoscope for receiving images from the lens.

According to a seventh aspect of the present invention, there is provided apparatus for inserting an instrument into a body cavity, the apparatus including a sheath having an inner chamber for accommodating the instrument and an outer chamber surrounding the inner chamber, wherein the outer chamber is provided with a protrusion to form a channel between the inner chamber and the outer chamber.

The channel may be for accommodating medical instruments or the like, or water or other fluid under pressure.

The protrusion may be defined between two substantially parallel welds between the inner chamber and the outer chamber. The protrusion may be provided with a re-entrant recess (a backwardly facing cut-out) at the proximal end of the sheath by removing an approximately semi-circular portion of the outer chamber. This reduces the height of the protrusion at the proximal end of the sheath, the height gradually increasing with distance from the proximal end of the sheath. Advantageously, the inner and outer sheaths may be temporarily bonded together at the proximal end of the sheath to further reduce friction as the sheath is inserted into the body cavity (so that the protrusion is only formed after the temporary bond breaks). The adhesive may be an adhesive that degrades and becomes less effective on contact with fluid within the body cavity to facilitate insertion of a medical instrument through the channel when the sheath is present in the body cavity.

According to an eighth aspect of the present invention, there is provided apparatus for inserting an instrument in a body cavity, the apparatus including a sheath having a chamber for accommodating the instrument and a channel extending along the chamber, thereby defining an enclosed space at least one hole in said chamber positioned within said enclosed space, means for temporarily applying fluid pressure to said enclosed space so as to cause the fluid to pass through the hole to build up pressure between the instrument and the chamber to facilitate removal of the instrument from the chamber.

According to an ninth aspect of the present invention, there is provided an endoscope (or other medical instrument) end cap having an aperture therethrough alighnable with a working channel of the endoscope, the end cap including a rim formation for abutting an edge region of said endoscope and for preventing the passage of the endoscope thought the aperture.

The body cavity mentioned above may be the body cavity of an inanimate object, or of the human or animal body.

The embodiment describes the apparatus used with cystoscopes. However, it should be understood that the invention applies to many types of instruments. The instruments may be elongate. The instrument may be a medical instrument. The instrument may be a medical telescope (or other viewing device). The invention applies to all endoscopes—e.g. ureteroscopes, bronchoscopes, colonoscopes, sigmoidoscopes, endoscopes, upper Gi scopes, ENTscopes etc.

### BRIEF DESCRIPTION OF THE DRAWINGS

For a better understanding of the present invention, embodiments will now be described by way of example, with reference to the accompanying drawings, in which:

FIG. 1 shows a perspective view of an endoscope and sheath apparatus in accordance with an embodiment of the invention;

FIG. 2 shows an exploded perspective view of the endoscope and sheath apparatus of FIG. 1;

FIG. 3 shows a side elevational view of the distal end of the sheath apparatus and the domed lens of FIG. 2, but in more detail;

FIG. 4 shows a side elevational view of the proximal end of the sheath apparatus and endoscope according to another embodiment of the invention;

FIG. 5 shows a perspective view of the sheath apparatus and endoscope according to a further embodiment of the invention in which the housing is partially cut away;

FIGS. 6A, 6B and 6C show the insertion of an endoscope within the sheath into a body cavity;

FIG. 7 shows a perspective view of the endoscope and sheath assembly, showing the inversion of the sheath during withdrawal of the endoscope from the sheath;

FIG. 8 shows an alternative embodiment of the invention in which the outer chamber of the sheath assembly includes a removable flap for facilitating the removal of the endoscope from the sheath;

FIG. 9 shows a side cross-sectional view of the distal end of an endoscope having an end cap according to an embodiment of the invention fitted thereto;

FIGS. 10A,10B and 10C show an alternative arrangement of end cap; and

FIG. 11 shows an end elevational view of the sheath according to a further embodiment of the invention in which an outer chamber is provided with a protrusion to form a working channel;

FIG. 12 shows a modification of the FIG. 11 embodiment in which a backwards facing recess is formed in the outer chamber to reduce friction when the sheath is inserted into a body cavity;

FIG. 13 shows a modification of the FIG. 12 embodiment in which adhesive is applied between the inner and outer chambers in the region of the recess to further reduce friction when the sheath is inserted into the body cavity;

FIG. 14A shows a perspective view of an alternative arrangement of the proximal end of the sheath;

FIG. 14B shows a side elevational view of the FIG. 14 arrangement;

FIG. 15A shows a perspective view of another arrangement of the proximal end of the sheath;

FIG. 15B shows a cross-sectional view of the FIG. 15A arrangement;

FIG. 16 shows an overhead plan view of the endoscope and sheath apparatus of FIG. 1;

FIG. 17A shows a cross-sectional view taken along lines A-A of FIG. 16;

FIG. 17B shows an enlarged view of area B of FIG. 17A;

FIG. 18A shows a perspective view of another embodiment of the sheath, showing a third chamber fitted over the proximal end of the sheath;

FIG. 18B shows the sheath of FIG. 18A but with the third chamber shrink fitted to the sheath;

FIG. 18C shows a side elevational view of the sheath of FIG. 18B being inserted into the urethera of a patient;

FIG. 19A shows a perspective view of another arrangement of a proximal end of the sheath in which an end cap having a central disc removed is fitted;

FIG. 19B shows the sheath of FIG. 19A into which an endoscope is inserted; and

FIG. 19C shows the sheath of FIG. 19A with multiple fibre channels.

In the drawings like elements are generally designated with the same reference sign.

### DETAILED DESCRIPTION OF EMBODIMENTS OF THE INVENTION

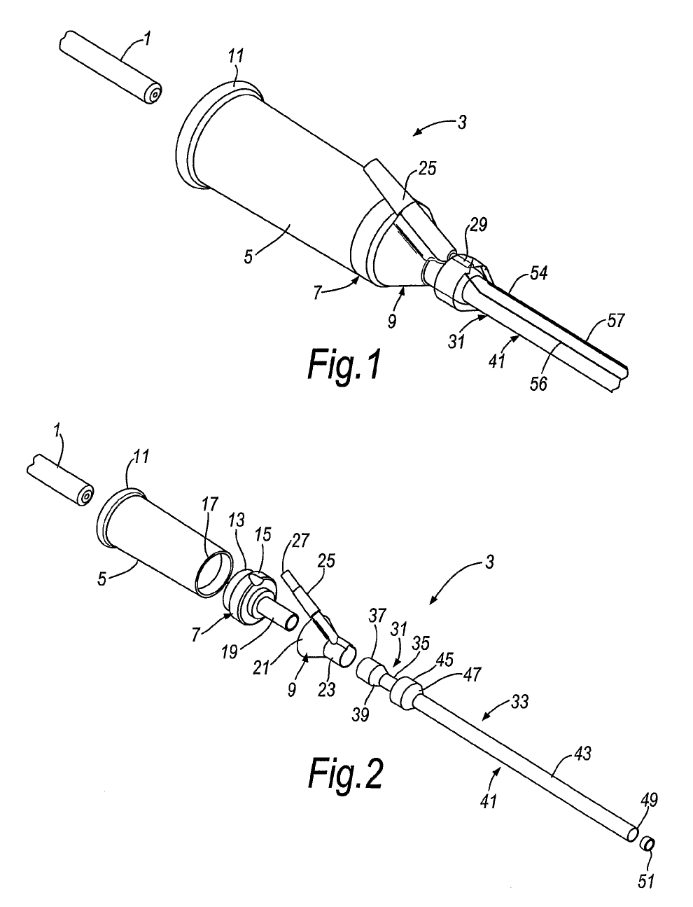

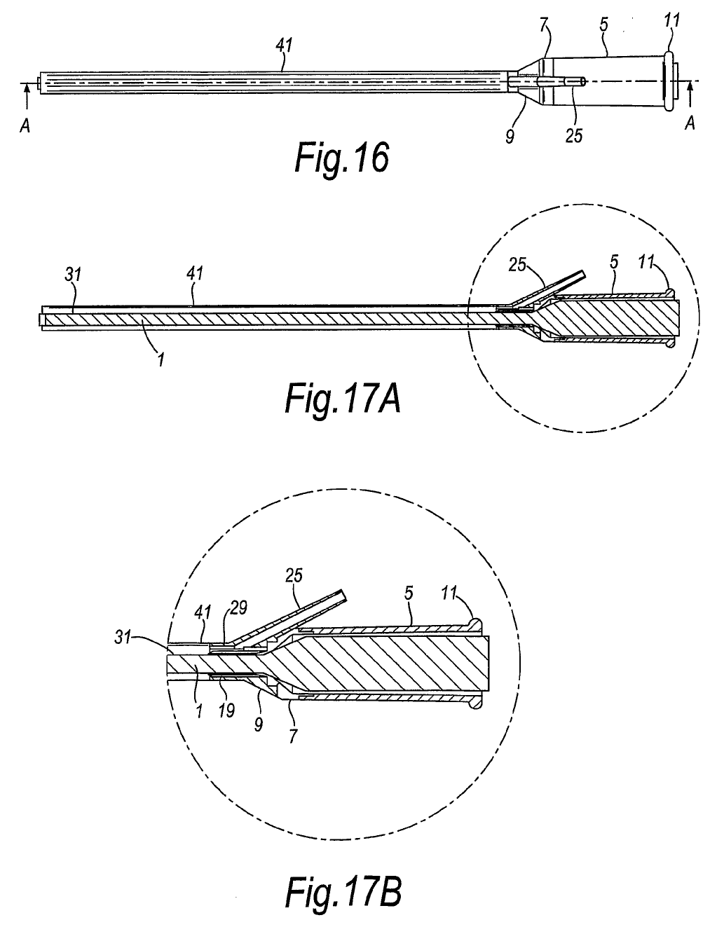

In the perspective view of FIG. 1 and the exploded perspective view of FIG. 2 the endoscope 1 is shown prior to insertion into the sheath apparatus 3 and a sheath 33. As also shown in FIG. 16,17A and 17B, the sheath apparatus 3 comprises a housing 5 formed of flexible material, an intermediate rigid moulding 7 and a rigid end moulding 9. The flexible housing 5 and rigid mouldings 7 and 9 are advantageously formed by a “twin shot” process which moulds the flexible material of housing 5 to the rigid polymers of mouldings 7 and 9. This allows the housing 5 to accommodate changes in diameter and shape of the endoscope 1.

The housing 5 comprises a hollow tube of circular cross-section, and may have a collar 11 formed at the endoscope-receiving end thereof. The intermediate moulding 7 includes an annular flange 13 which fits inside the internal diameter of the housing 5 so that the collar 15 of the intermediate moulding 7 abuts against the end face 17 of the housing 5 at the end opposite to the collar 11. The intermediate moulding 7 further comprises a tube 19 of circular cross-section sufficient to accommodate the endoscope 1 therein, and which extends from the collar 15. The tube 19 comprises part of the cylindrical passageway through the intermediate moulding 7 for accommodating the endoscope 1.

The end moulding 9 comprises a hollow frusto-conical portion 21, the smaller diameter part of which terminates in a hollow tube 23 of cylindrical cross-section, sized to accommodate the tube 19 of the intermediate moulding 7. A port 25 having an inlet 27 is in fluid communication with an outlet 29 on the exterior of the tube 23 of the end moulding 9.

An inner chamber 31 of the endoscope sheath 33 comprises an elongate hollow tubular portion 35 of circular cross-section—for example of 6 mm diameter. The portion 35 is coupled to a tubular collar 37 by a frusto-conical intermediate portion 39. The collar 37 has an internal diameter such that it fits inside the tube 23 of the end moulding 9 and over the tube 19 of the intermediate moulding 7 and is able to receive the endoscope 1.

The sheath 33 further comprises an outer chamber 41. The outer chamber 41 comprises an elongate hollow tubular part 43 which has a constant circular cross-section and an internal diameter sufficient to accommodate the elongate tubular portion 35 of the inner chamber 31—for example 7 mm diameter. The outer chamber 41 further comprises a collar 45 of enlarged cross-section, for accommodating the collar 37 of the inner chamber and for fitting over the tube 23 of the end moulding 9. The arrangement is such that fluid from the inlet 27 of the port 25 passes to the outlet 29 and from there into the elongate tubular part 43 of the outer chamber 41 via the collar 45. The collar 45 is coupled to the elongate tubular part 43 by frusto-conical intermediate part 47.

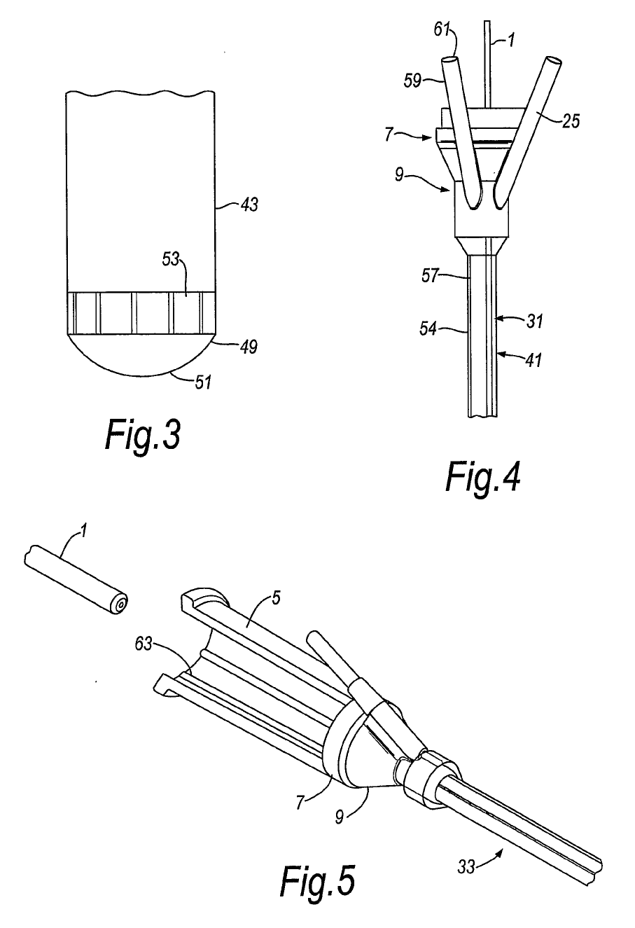

The distal end of the elongate tubular part 43 of the outer chamber 41 terminates in an opening 49. An enlarged view of the distal end of the part 43 is shown in FIG. 3. The opening 49 is closed by a dome shaped lens 51 that provides substantially optically perfect 180° panoramic vision. The dome shaped lens 51 may cover the whole of the tip end or just part of it. Where the domed lens 51 only covers part of the tip end the remainder of the tip end would be substantially flat. The domed lens 51 is bonded to the interior surface of the part 43 using UV curable adhesive of a type suitable for secondary sterilisation—for example, Locktite 3341. The adhesive bonds the collar 53 of the domed lens 51 to the interior surface of the part 43. The lens may be shaped to provide other degrees of view, for example zero degrees, or 30 degrees or 110 degrees. It may also be flat so as not to distort the image received by the endoscope.

As shown also in FIG. 4, a separate channel 54 (e.g. a “working channel”) may be formed between the inner fluid chamber 31 and the outer fluid chamber 41 by heat welding the inner fluid chamber 31 to the outer chamber 41 along substantially parallel lines 56 and 57 (see FIG. 1). The heat welded lines 56 and 57 extend along the elongate parts 35, 43 and also along the frusto-conical parts 39, 47 of the fluid chambers 31, 41. The joint at lines 56 and 57 may alternatively be made by application of another energy form. The channel 54 formed between the inner and outer chambers 31, 41 may receive medical instruments such as forceps. The channel 54 may be fed by secondary port 59, through the inlet 61 of which the medical instrument is inserted.

A plurality of such channels may be provided. The type, thickness and size of the working channels may be varied to accept secondary tubes which may not distort due to their particular use.

The longitudinal welds along lines 56 and 57 also help insertion of the sheath 33 as the welds increase the longitudinal stiffness of the sheath 33, which reduces the tendency of the sheath 33 to wrinkle, thereby reducing patient discomfort and the force required to push the scope 1 in—as a smooth, even surface offers less resistance than a wrinkled surface.

Also, the walls of the inner and outer chambers 31,41 may vary in thickness to increase channel puncture resistance.

As mentioned above, in the arrangement shown in FIGS. 1 and 2 different sizes of endoscope 1 are accommodated by a combination of the rigid plastic mouldings 7 and 9 and “twin shot” moulding of the flexible housing 5. FIG. 5 shows an alternative arrangement in which the housing 5 is provided with a plurality of equi-spaced longitudinally extending webs 63 extending in the direction of insertion of the endoscope 1. The webs 63 formed inside the flexible housing 5 maintain sufficient contact with the endoscope 1 during handling for endoscopes of varying sizes.

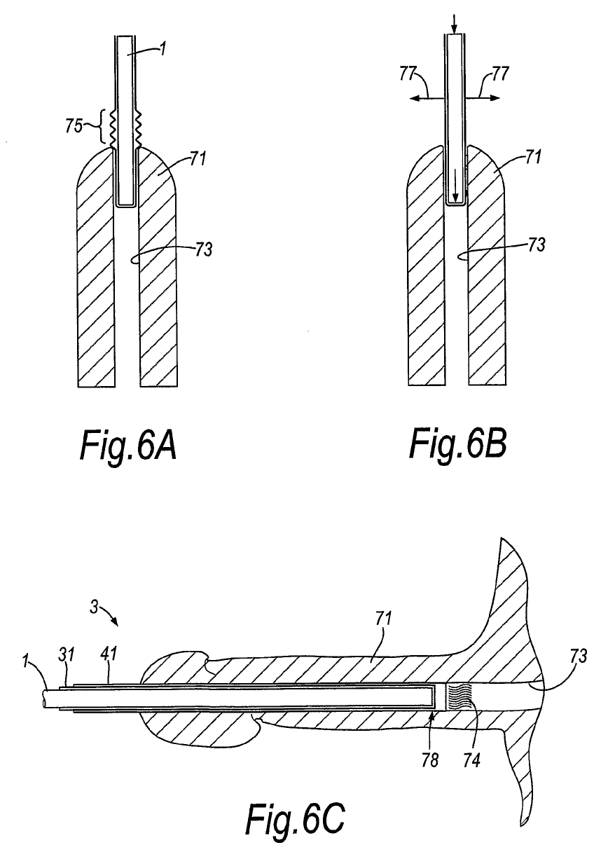

FIGS. 6A,6B and 6C show the endoscope 1 surrounded by the inner chamber 31 (FIG. 6C) and the outer chamber 41 (FIG. 6C) as it is inserted into the penis 71 (FIGS. 6A,B,C) of a patient. FIGS. 6A and 6B show only one fluid chamber. Two fluid chambers are present but only one is shown for clarity.

Because the opening of the urethra 73 of the penis 71 is small, this causes the material of the first and outer chambers 31, 41 to fold or wrinkle as shown at 75 as the endoscope 1 is pushed into the orifice 73—FIG. 6A. The wrinkling of the material of the inner and outer chambers 31, 41 obstructs further insertion of the endoscope 1 and is painful for the patient. According to a feature of this embodiment of the invention, fluid pressure is applied between the inner chamber 31 and the outer chamber 41 which imposes a form of rigidity both radially and longitudinally. Thus, the outer chamber 41 expands in the direction of arrow 77 (its diameter increases), which gently opens the urethra 73, removes the wrinkling 75 and allows for further, relatively painless, insertion of the endoscope 1—FIG. 6B.

The fluid pressure can be applied from a head of saline solution in a “giving set” which exerts a pressure of approximately 0.2 bar on the internal surface of the outer chamber 41. The pressure applied can be increased by the operator squeezing the giving set. Further, the pressure applied could be varied periodically in order to temporarily increase the diameter of the outer chamber 41, and hence the urethera 73, to assist insertion of the endoscope 1 when the pressure is subsequently reduced.

FIG. 6C shows the sheath, constructed of a 6 mm diameter inner chamber 31 and an open ended 7 mm diameter outer chamber 41 enclosing the endoscope 1. It is assumed that during insertion into the penile urethra 73, the forward motion of the sheath assembly 3 will be constricted/gripped by the urethra 73. Without this constriction/gripping the saline solution 74 would exit under gravity through the open end of the annulus between inner chamber 31 and outer chamber 41 at point 78. As the assembly 3 progresses through the urethra 73 the natural constriction/gripping will cause the outer chamber 41 to be squeezed against the inner chamber 31 and the endoscope 1 providing for a pressure build up from the head of saline solution. This pressure can be increased or decreased by squeezing the Giving Set or introducing an oscillating wave pattern by using a peristaltic pump or similar. The pressure build up at the entry of the urethra 73 gently opens the urethra 73 and assists insertion of the assembly 3.

As an alternative to, or in addition to, relying on the natural constriction/gripping caused by the body cavity to build up fluid pressure, the outer chamber 41 may be formed to restrict the flow of saline solution from its distal end. For example, the diameter of the outer chamber 41 may be reduced at its distal end, or the distal end may be partially closed, to inhibit the flow of saline solution out of the outer chamber 41.

Withdrawing endoscope 1 from the inner chamber 31 and the outer chamber 41 can be problematic due to friction between the endoscope and the inner chamber 31, and friction between the inner chamber 31 and the outer chamber 41. In accordance with the embodiments, this problem can be mitigated in several ways.

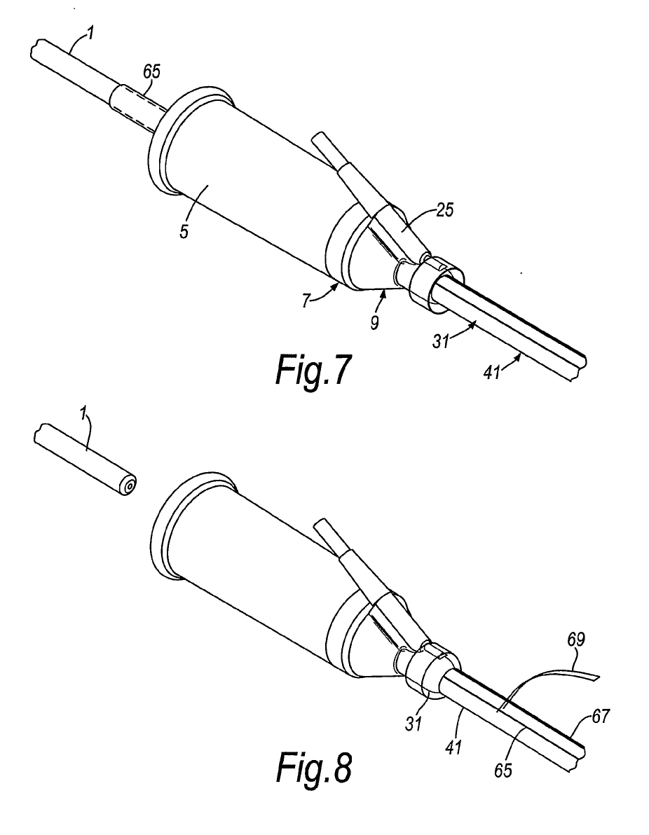

Firstly, as shown in FIG. 7, the friction between the endoscope 1 and the inner chamber 31, and the corresponding friction between the inner chamber 31 and the outer chamber 41, can be used to invert the first and outer chambers as the endoscope 1 is withdrawn, so that the first and outer chambers 31, 41 are inverted with the endoscope, as shown at 65 in FIG. 7.

FIG. 8 shows an alternative solution. In the FIG. 8 embodiment, the first (inner) fluid chamber 31 has an equal or greater diameter than the second (outer) fluid chamber 41, or the fluid chambers 31, 41 have equal diameters. A failure mechanism is incorporated into the outer chamber. For example, this failure mechanism may comprise two substantially parallel lines of weakness 65, 67 extending longitudinally along the outer chamber 41 which allows a strip of material 69 of the outer chamber 41 to be torn from the surrounding material. This releases the inner chamber 31 and allows the endoscope 1 to be freely withdrawn.

The inner chamber 31 may be folded, pleated, or twisted or flared (the folds or pleats extending longitudinally) when it is accommodated within the outer chamber 41. This means that the outer sheath with its built in failure mechanism when intact keeps the diameter of the whole sheath apparatus 3 constant, but when the failure mechanism is broken (strip 69 removed) the inner chamber 31 which covers the endoscope 1 expands to its larger diameter, allowing the endoscope 1 to be withdrawn from it with ease. A major problem with known sheaths is that they tend to grip the endoscope 1 very tightly and it is therefore difficult to remove the endoscope from the sheath. By placing a fold/pleat in the larger diameter inner chamber 31 the problem of endoscope 1 withdrawal from the sheath system is overcome. The fold/pleat allows the inner chamber to expand when the failure mechanism is broken so that it does not grip the endoscope 1.

Alternatively to, or additionally to, the arrangement described in relation to FIG. 8, the inner chamber 31 may be provided with a failure mechanism to facilitate removal of the endoscope 1 therefrom and/or the outer sheath 41 may be provided with folds or pleats to allow its expansion circumferentially.

Thirdly, the outer chamber 41 and/or the inner chamber 31) could include, or could be coated with, a friction reducing polymer to reduce friction between the outer chamber 41 and the body cavity and/or between the inner chamber 31 and the endoscope 1.

The friction reducing polymer may contain phosphorycholine (PC). PC is zwitterionic: it has a positive and negative charge on the same molecule but is electrochemically neutral overall. PC therefore has high polarity and consequently a natural affinity to water. A substrate that includes PC material is surrounded by molecular layers of water, which effectively forms a barrier over the substrate. The barrier provides a “non-stick” biological surface that resists protein and cell adhesion.

By coating the outer chamber 41 or the inner chamber 31 with a polymer that contains PC materials, the friction between the outer chamber 41 and the body cavity and/or between the outer chamber 41 and the inner chamber 31 and/or between the inner chamber 31 and the endoscope 1 may be significantly reduced.

Because PC materials also resist bacterial adhesion, applying a polymer including PC material to the outer chamber 41 and/or the inner chamber 31 reduces the adhesion of bacterial materials to the endoscope sheath 33. This can be particularly advantageous to reduce potential transportation of bacteria at the end of the penis into the bladder during the sheath insertion process.

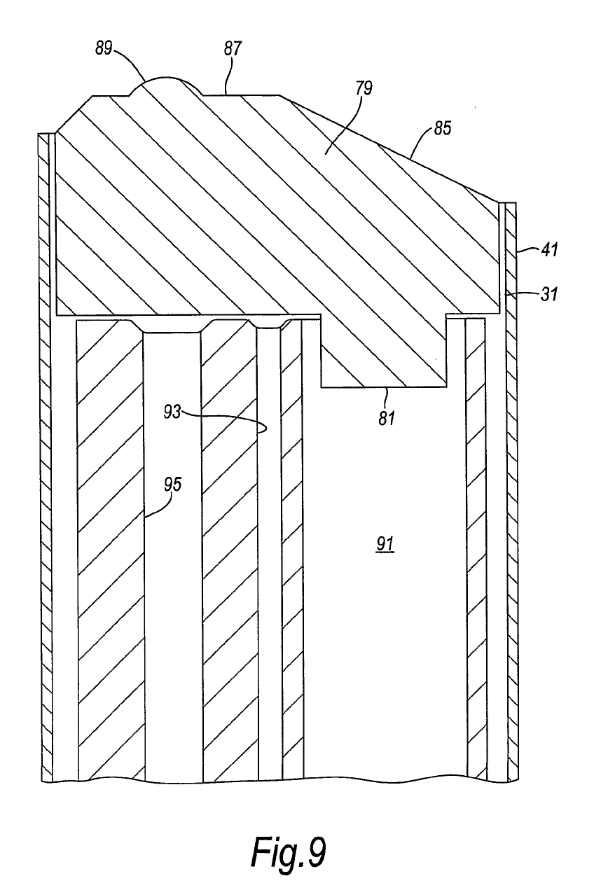

FIG. 9 shows an alternative arrangement of the endoscope 1, the inner chamber 31 and outer chamber 41 at the distal end thereof. The domed lens 51 of the outer chamber 41 of the FIG. 3 embodiment is replaced with an end cap 79 mounted on the endoscope 1. The end cap 79 includes a protrusion 81, for example of cylindrical cross-section, which passes into a working channel 91 of the endoscope 1. This provides for correct alignment between the end cap 79 and the endoscope 1 and reduces wandering of the endoscope 1 within the chambers 31 and 41, and improves the images captured by the endoscope 1.

The endoscope 1 of this embodiment includes, in addition to the working channel 91, a channel 93 for receiving a light cable, such as a fibre optic cable which allows illumination of the body cavity, and a channel 95 in which a camera may be housed.

The end cap 79 (see FIG. 9) is formed from transparent material for allowing the camera or optics in channel 95 of the endoscope 1 to view the body cavity. The end face 87 of the end cap 79 has a domed lens 89 positioned in alignment with the camera/optics channel 95 of the endoscope 1.

The end cap 79 may comprise a bevelled edge 85.

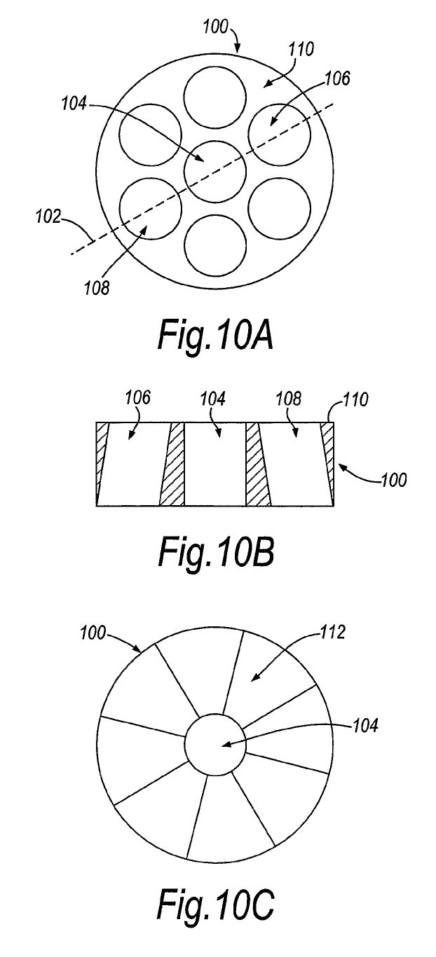

FIGS. 10A,B and C show an alternative end cap 100. FIG. 10A shows an end on view of the end cap 100. FIG. 10B shows a cross section of the dashed line 102 of FIG. 10A. FIG. 10C shows an alternative arrangement for the division of chambers in the end cap 100.

With a smaller diameter endoscope (e.g. 2 mm diameter) placed down the central lumen 104 in FIG. 10C, this leaves the potential for multiple other access channels 106,108 around it. The central channel 104 may have a plain window or a lens, alternatively any of the channels 104,106,108 could accommodate the endoscope 1 with an appropriate window/lens at the end. The other channels would be open allowing the passage of instruments or fluids.

The cross section of an end cap 100 shown in FIG. 10B shows that the channels 104,106,108 may run straight through the end cap 100 at 90 degrees to the end surface 110 (e.g. channel 104) or may run at another angle giving a slanted or oblique channel (e.g. channels 106 and 108). These angled channels 106,108 have the advantage that instruments (e.g. laser fibres etc.) would be diverted away from the central field of view once inside the lumen to be inspected. For example, if multiple laser fibres were passed down these channels this would have the effect of having a much larger field of ablation provided by the laser than a single fibre alone.

The laser fibres are typically 200 μm in diameter and the channel of a flexible ureteroscope for example is 1.2 mm. This means a bundle of fibres could be passed down the channel and as they emerged from the channels 106,108 in the end cap 100 diverge from one another. This would provide a relatively larger area over which the fibres would have their effect. When used with an endoscope with an integral lumen the holes may be positioned so that they are only present over the lumen of the endoscope. Ideally the end cap 150 is kept as thin as possible so as not to reduce the visual clarity of the image seen on the scope. However, when channels 104,106,108 are placed in the end cap for laser fibres this section of the end cap may need to be thicker than the rest of the end cap. This may take the form of a protrusion which may project forward, out from the surface of the end cap away from the face of the scope or the protrusion may face backwards entering the working channel of the scope. In addition instead of covering the whole of the surface of the tip of the scope the end cap 100 may just partially cover it so that it only fits over the working channel of the scope. This may be circular in shape or cresenteric or any shape.

In FIG. 10C the channels 112 surrounding the central channel 104 are segmented.

When the end cap is attached to the sheath this keeps it in position on the endoscope. Alternatively the end cap 100 may be secured in position at the tip of the scope with a ring or horseshoe type clip when the sheath is not used. Alternatively a fine wire which may pass along the working channel or the outside of the scope may have the end cap secured to it so that the end cap can be moved up and down the working channel or alongside the scope as required.

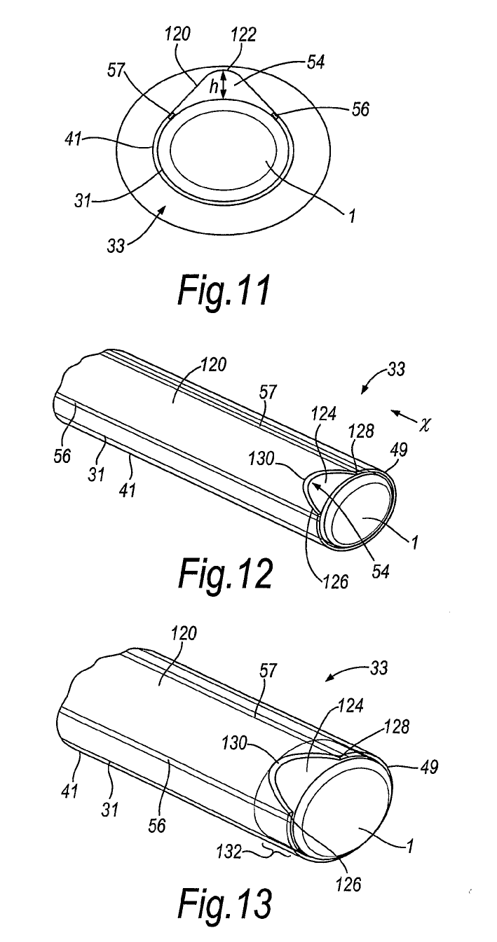

FIG. 11 shows a further embodiment of the invention in which the circumference of at least part of the outer chamber 41 is reduced by forming an offset or protrusion 120 in the outer chamber 41 between substantially parallel welded lines 56 and 57 that form a channel 54 between the inner and outer chambers 31,41. By forming the channel 54 by providing the protrusion 120 in the outer chamber 41, space is provided between the outer chamber 41 and the inner chamber 31 to accommodate a medical instrument, such as biopsy forceps or similar. An endoscope 1 (or other instrument) may be accommodated in the inner chamber 31. A problem with the arrangement shown in FIG. 11 is that the protrusion 120 is present even when no medical instrument is present in the working channel 54. The shape of the protrusion means that a point of maximum friction 122 occurs, where the protrusion contacts the body cavity at the point where the height h of the protrusion 120 is at its maximum. This tends to cause difficulties in inserting the sheath 33 into the body cavity.

FIG. 12 shows a modification to the FIG. 11 embodiment in which a protrusion 120 like that shown in FIG. 11 is present in order to provide the working channel 54. However, at the opening 49 of the outer chamber 41, at the proximal end of the sheath, an approximately semi-circular portion of the outer chamber 41 is omitted to form a generally concave re-entrant (backwards facing) recess 124 in the outer chamber 41. The re-entrant edges 126,128 of the recess 124 extend substantially to the heat welds 56 and 57, respectively. The distal edge 130 of the recess 124 is provided midway between the heat welds 56 and 57. The protrusion 120 therefore tapers towards the opening 49 at the proximal end of the sheath 33.

By providing the recess 124, the height h that the protrusion 120 extends above the inner chamber 31 increases gradually in the direction x from the opening 49. This reduces friction as the sheath 33 is inserted into the body cavity as the maximum height h of the protrusion 120 only has to be accommodated by the body cavity gradually as it is inserted. The point of maximum friction 122 is therefore distributed over a larger part of the body cavity.

FIG. 13 shows a further embodiment of the invention, which is a modification of the FIG. 12 embodiment. In the FIG. 13 embodiment the region 132 of the ends of the inner sheath 31 and the outer sheath 41 adjacent to the opening 49 are welded or adhered together prior to deployment of the sheath 33 in the body cavity. Adhesive is applied between the interior surface of the outer cavity 41 and the exterior surface of the inner cavity 31 in the region 132. This adhesive may be a medical grade water soluble low adhesion adhesive which quickly loses its adhesion when in contact with fluids present in the body cavity.

The arrangement of FIG. 13 allows the height h of the protrusion 120 to be minimal in the region 132 because the material of the inner chamber 31 and the outer chamber 41 are adhered together by the adhesive in this region. The height h of the protrusion 120 then gradually increases on the opposite side of the region 132 to the opening 49. This further reduces friction when the sheath 33 is inserted into the body cavity.

The adhesive may be such that the fluids present in the body cavity cause the adhesive in the region 132 to degrade so that the working channel 54 can be opened in the region 132 to allow medical forceps or other instruments to pass along the working channel 54.

The adhesive in the region 132 may be applied to any embodiment of the invention including those where no recess 124 or protrusion 120 is provided.

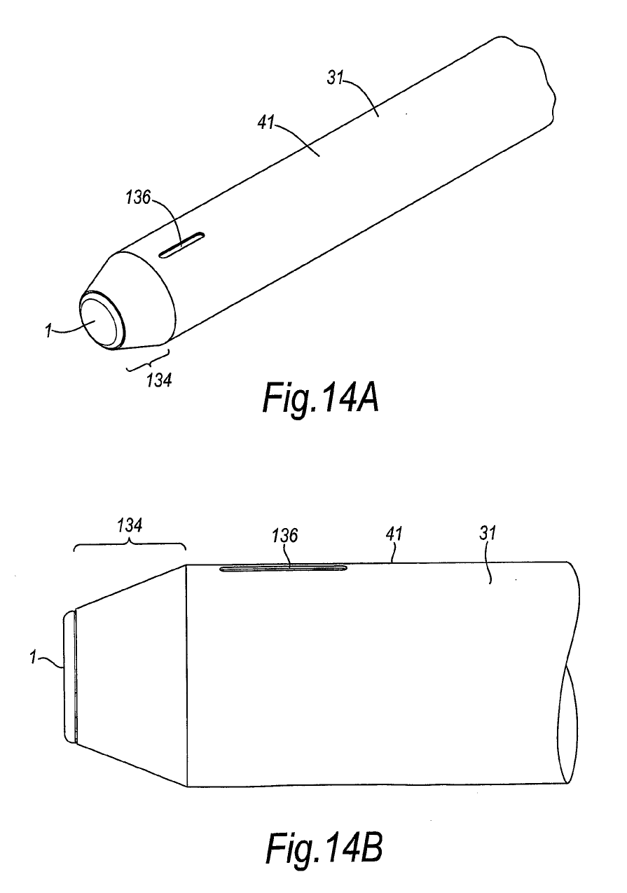

FIGS. 14A and 14B show a further embodiment of the invention in which the circumference of the outer chamber 41 is reduced at the proximal end by tapering the outer chamber 41 in region 134.

This arrangement has the advantage of allowing a larger diameter outer channel 41 to be used that becomes narrower towards the proximal end that is inserted into the patient reducing discomfort and frictional resistance at insertion.

In addition the inner chamber 31 may be tapered at its proximal end. This has the advantage of allowing a predominantly larger diameter inner chamber 31 to be used which will be easier to remove from the endoscope 1 than one which is close fitting to the endoscope 1 over its whole working length. Furthermore the inner and/or other chambers 31,41 may be flared outwards at their distal ends where they are attached to the housing 5. This will allow easier fitting to the moulding 9, reducing stretching of the sheath material at this connection point.

The outer sheath chamber 41 may have a slit 136 in it allowing the passage of fluid or instruments. The slit 136 may be straight (as shown), curved, diagonal, cresentic or some other shape. The slit 136 may be complete or partial. When the slit 136 is partial it may have lines of weakness incorporated into it—this will allow the free passage of fluid and when an instrument is passed down, the areas of weakness will be broken opening up the full slit. Alternatively the slit 136 may be partially closed by means of a temporary adhesive.

The tapered inner/outer chambers 41,31 may be combined with the FIG. 11,12 or 13 embodiments.

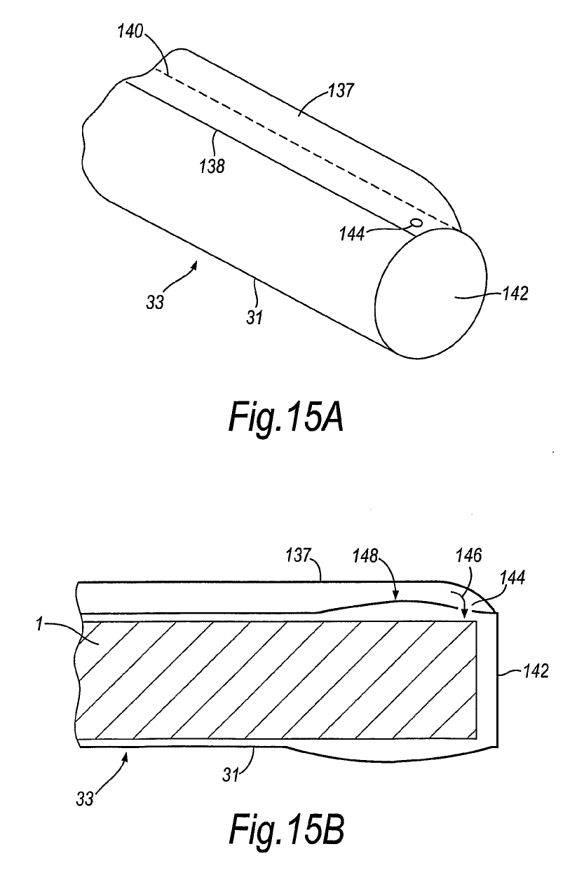

FIGS. 15A and 15B show a further embodiment of the invention. In this embodiment the sheath 33 comprises a chamber 31 which accommodates the endoscope 1. The chamber 31 is provided with a separate channel 137. This channel 137 may be formed by heat welding flexible material to the chamber 31 along substantially parallel lines 138 and 140. The join at lines 138 and 140 may alternatively be made by application of another energy form. The channel 137 may surround only a portion of the circumference of chamber 31. The channel 137 may be formed from an outer fluid chamber 41 which completely surrounds the inner fluid chamber 31, as in the preceding embodiments, or the channel 137 may be formed from material which extends only between the parallel lines 138 and 140, as shown in FIGS. 15A and 15B.

The fluid chamber 31 and channel 137 at the proximal end are sealed to end cap 142. A hole 144 is provided in the fluid chamber 31 to allow fluid to pass from the channel 137 into the fluid chamber 31.

For example, sterilized liquid may be pumped along the channel 137 under pressure. The liquid passes through the hole 144 as indicated by arrow 146 which pushes the chamber 31 away from the endoscope 1, as shown by the bulging of the chamber 131 at point 148 in FIG. 15B. This frees the end of the endoscope 1 from the chamber 31 and makes it easier to withdraw the endoscope 1.

More than one hole 144 may be provided. More than one channel 136 may be provided.

In an arrangement where an outer chamber 41 is provided, the outer chamber 41 is sealed at its proximal end to the end cap 142 of the inner chamber 31 and the inner chamber 31 has perforation(s) 144 at its proximal end and or along its length. These perforations 144 would generally be present between two longitudinal welds 138,140. When the channel space 136 between the two longitudinal welds 138,140 is pressurised with a gas/fluid medium, this will pass through the perforations 144 to the space between the inner chamber 31 and the scope 1. This causes the inner chamber 31 to be pushed off the surface of the scope 1, allowing easy withdrawl of the scope 1 from the inner chamber 31. In addition one or more additional layers of material/chambers may be added to the outside of this arrangement to create a working channel.

An end cap 79,100,142 may be provided with a channel for allowing therethrough fluid under pressure to facilitate removal of the endoscope from the sheath 33. The channel allows water to pass between the endoscope 1 and the inner chamber 31.

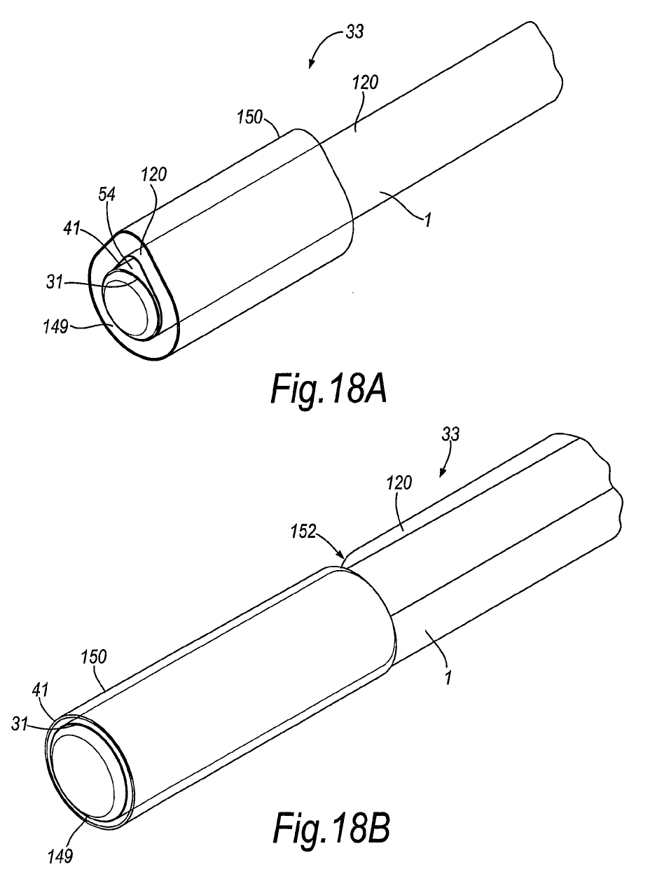

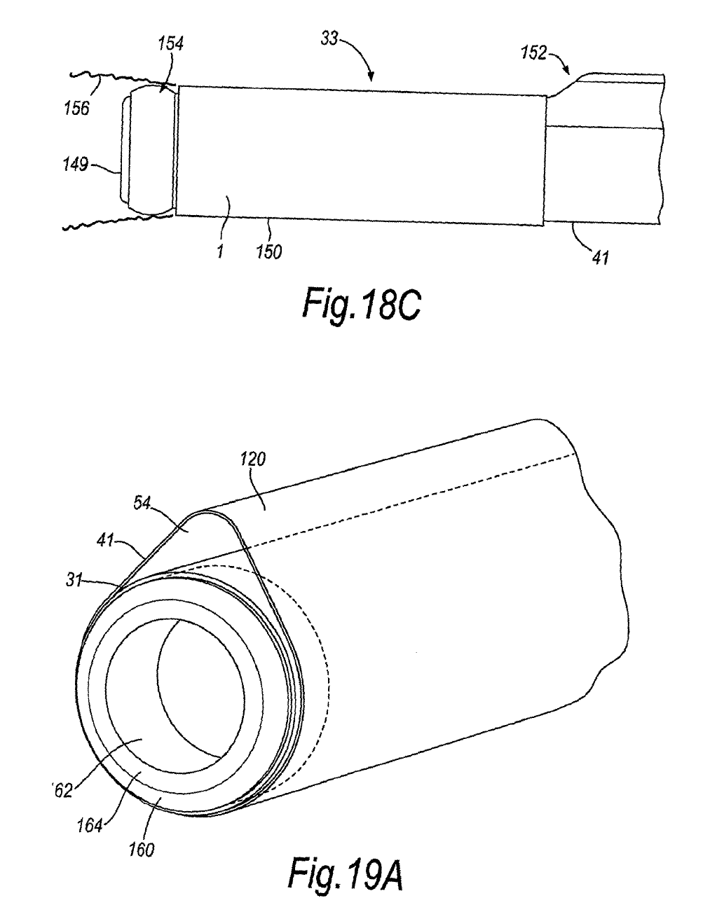

FIG. 18A shows a further embodiment of the invention in which, like the FIG. 11 embodiment, an offset or protrusion 120 is formed in the outer chamber 41 to provide a channel 54 between the inner and outer chambers 31,41. By forming the channel 54 by providing the protrusion 120 in the outer chamber 41, space is provided between the outer chamber 41 and the inner chamber 31 to accommodate a medical instrument, such as biopsy forceps or similar. An endoscope 1 (or other instrument) may be accommodated in the inner chamber 31, which is closed by end cap 149.

In view of the function of the sheath 33 to completely encapsulate an endoscope 1 to maintain sterility of the scope 1, the diameter of the sheath 33 is marginally greater than the scope 1. The larger the diameter of the sheath 33, the more difficult insertion into the body cavity is. This difficulty is exacerbated by the elliptically shaped channel 54 on the periphery of the sheath 33. In combination the effect of these greater diameters can lead to discomfort for the patient during an investigation due to the end of the outer chamber 41 abrading the inner lining of the urethra. In accordance with this embodiment of the invention, this discomfort can be minimised or even eliminated by the addition of a third chamber 150 over the end of the sheath to provide a smooth contour 152 as shown in FIG. 18B, although the third chamber 150 can be fitted at the proximal end of the sheath 33 or throughout its length.

In FIG. 18A the third chamber 150 is shown with a much larger diameter than the outer chamber 41. In use, the third chamber 150 has a diameter the same as or smaller than the outer chamber 41. The third chamber 150 may be fitted over the outer chamber 41 as shown in FIG. 18A, and its diameter may then be reduced by shrinking, as shown in FIG. 18B. The third chamber 150 tapers the end of the outer chamber 41 to minimize its protrusion 120 and create a smooth contour 152.

As shown in FIG. 18C, when fitted in place by shrinking, welding or adhering to the outer chamber 41 the third chamber 150 creates a smooth entry function by virtue of the smooth contour 152. This can be enhanced by modifying the end cap 149 to have a form as shown in FIG. 18C to have a domed circumference 154. The domed circumference 154 is of substantially the same circumference as that of the third chamber 150 in use. The domed circumference 154 acts as a form of deflector to ease the lining of the urethra 156 away from sheath 33 during entry. The domed circumference 154 may be formed by the glue used to adhere the end cap 149 to the sheath 33.

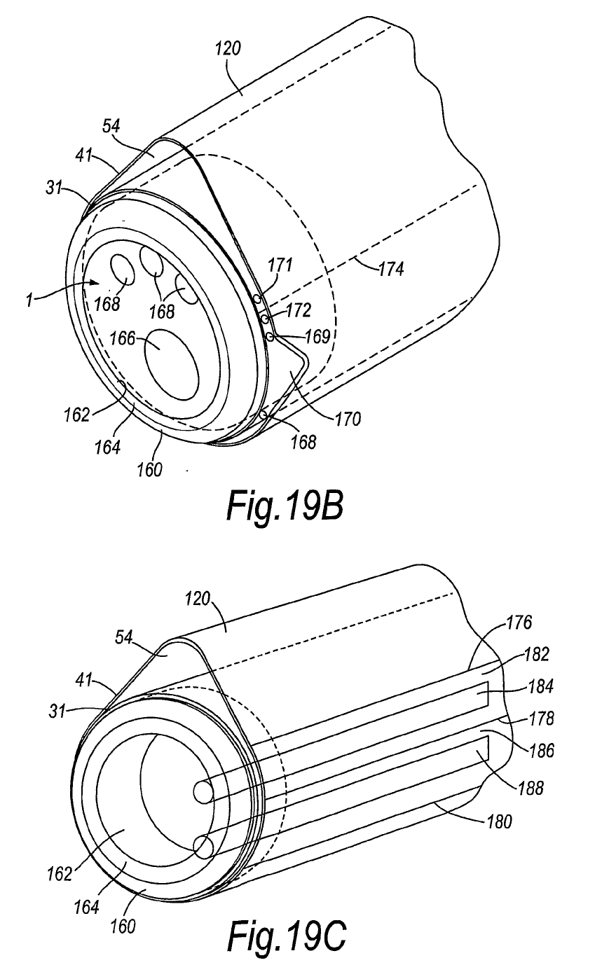

FIG. 19A shows a further embodiment in which the sheath 33 has a protrusion 120 like the FIG. 11 embodiment to provide a channel 54. An end cap 160 is fitted to the proximal end of the inner chamber 31. The end cap 160 has a central circular aperture 162 therethrough. The end cap 160 defines an annular rim 164. As shown in FIG. 19B, when endoscope 1 is inserted in the sheath 33, the proximal end of the endoscope 1 abuts the annular rim 164 of the end cap 160. The aperture 162 and the rim 164 may be of different form—and are not necessarily circular.

The endoscope 1 of this embodiment includes, a working channel 166 and three further relatively small channels 168.

In continuous flow cystoscopy (which is the norm in rigid scopes), irrigation is conventionally performed by passing fluid in down the channel 54 and out up the working channel 166 in the scope 1. This would allow clearer vision and larger bladder tumours to be taken on with a laser.

This aspect can be adapted for all flexible and potentially some rigid scopes.

Flexible ureteroscopes 1 vary from 7.5-9 F (2.5-3 mm diameter) and are around 60 cm long, they have a 1.2 mm working channel. A problem arises when an instrument is passed into the working channel 166. Fluid flow through the working channel 166 and thus vision is decreased. Higher pressure can be applied to bypass the instrument in order to overcome this problem, but is not ideal.

In this embodiment, the inner chamber 31 and outer chamber 41 form a multi-layered septated sheath that fits over the scope 1. Septations 168 and 169 may be formed by welding the inner chamber 31 to the outer chamber 41. The septations 168 and 169 provide an additional channel 170. This additional channel can be for allowing irrigation fluid to pass up the channel to allow the working channel 166 to be used for instruments or suction by virtue of the aperture 162 in the end cap 160. Alternatively, the additional channel 170 can be used for instruments or suction.

A mechanism for maintaining the sheath 33 gently stretched on the scope 1 would be advantageous to stop it concertinaing, and this is provided by the rim 164. These fine scopes are delicate and care is required not to restrict their movement by a sheath that is too tight.

Lasers are used to remove tumors. Laser fibers for carrying radiation are typically 200 microns in diameter. In accordance with this embodiment, tightly welded septations 171 and 172 are provided between the inner chamber 31 and the outer chamber 41 to form a channel to accommodate one laser fiber 174, so it cannot move from side to side in the channel. A laser fiber may alternatively or additionally be accommodated in the additional channel 170.

FIG. 19C shows a further embodiment in which three welds 176,178 and 180 are made between the inner chamber 31 and the outer chamber 41 which form a first channel 182 for a first laser fibre 184 and a second channel 186 for a second laser fibre 188. The laser fibres 184,188 cannot move from side to side in the channels 182,186.

Currently flexi ureteroscopes cost around £15,000 each. They last between 20-40 uses and then must be replaced due to damage. Most of the damage is caused by laser fibers scratching the working channel. With the laser fibers on the outside of the scope in the sheath septations not only can several fibers be inserted at once, but the scope working channel is not damaged. This allows scopes to last longer and tumors would be broken more quickly allowing larger tumors to be dealt with ureteroscopically.

In any embodiment of the invention, multiple channels may be formed around the circumference between chambers 31 and 41, and each of these channels could be used for instruments, irrigation or other purposes.

1. Apparatus for inserting an instrument in a body cavity, the apparatus including:
a sheath having an inner chamber for accommodating the instrument and an outer chamber surrounding the inner chamber, thereby defining an enclosed space between the inner chamber and the outer chamber; and
means for temporarily applying fluid pressure to said enclosed space so as to expand the outer chamber to facilitate insertion of the instrument in the body cavity

2. The apparatus of claim 1, wherein said pressure applying means causes the outer chamber to expand radially outwardly.

3. The apparatus of claim 1, wherein said pressure applying means causes the outer chamber to expand longitudinally.

4. The apparatus of claim 1, wherein the expansion of the outer chamber causes the body cavity to expand.

5. The apparatus of claim 1, wherein the pressure applying means is operable to apply pressure of substantially 0.2 bar.

6. The apparatus of claim 1, wherein the pressure applying means comprises a giving set.

7. The apparatus of claim 1, wherein said pressure applying means applies pressure by means of saline solution in said enclosed space.

8. Apparatus for applying an instrument to a body cavity, the apparatus including a sheath having an inner chamber for accommodating the instrument and an outer chamber surrounding the inner chamber, wherein the inner chamber is expandable to have a diameter equal to or greater than the diameter of the outer chamber, and wherein the outer chamber incorporates at least one line of weakness for facilitating release and expansion of the inner chamber and withdrawal of the instrument from the inner chamber.

9. Apparatus according to claim 8, wherein the inner chamber incorporates at least one line of weakness.

10. Apparatus for inserting imaging means into a body cavity, the apparatus including a sheath for accommodating the imaging means, wherein said sheath includes at its proximal end a convex lens for use by said imaging means in obtaining images of said body cavity.

11. The apparatus of claim 10, wherein the said lens provides substantially 180° panoramic vision of the body cavity.

12. The apparatus of claim 10 or 11, wherein said lens is bonded to said sheath by a UV adhesive suitable for secondary sterilisation.

13. Apparatus for inserting an instrument into a body cavity, the apparatus including:
a sheath for accommodating the instrument;
a flexible housing for receiving the instrument;
a relatively rigid intermediate part between said housing and said sheath for guiding the instrument from said housing to said sheath.

14. The apparatus of claim 13, wherein the housing and intermediate part are formed by a twin shot moulding process.

15. The apparatus of claim 13, wherein the housing includes a plurality of webs on the internal surface thereof for guiding the instrument therealong.

16. The apparatus of claim 13, operable to accommodate multiple sizes and shapes of instrument.

17. The apparatus of claim 1, including phosphorycholine (PC).

18. A viewing device end cap including a lens and a formation for mechanically fitting the end cap to the viewing device such that the lens is aligned with a part of the viewing device for receiving images from the lens.

19. The viewing device end cap of claim 18, in combination with a sheath for accommodating the end cap and the viewing device.

20. A medical instrument end cap including an instrument channel and further channels for allowing the passage of further instruments therethrough or for allowing therethrough fluid under pressure to facilitate removal of a sheath arrangement from an associated instrument.

21. The end cap of claim 20, wherein the further channels extend obliquely with respect to the instrument channel.

22. The end cap of claim 20, wherein the further channels are adapted to receive laser fibres or other instruments.

23. The end cap of claim 20, in combination with a sheath for accommodating the end cap and the viewing device.

24. Apparatus for inserting an instrument into a body cavity, the apparatus including a sheath having an inner chamber for accommodating the instrument and an outer chamber surrounding the inner chamber, wherein the outer chamber is provided with a protrusion to form a channel between the inner chamber and the outer chamber.

25. The apparatus of claim 24, wherein the proximal end of the outer chamber includes a recess region which forms the proximal end of the protrusion for tending to reduce friction during the insertion of the sheath into the body cavity.

26. The apparatus of claim 24, wherein the inner chamber and the outer chamber are adhered to one another at the proximal end thereof.

27. The apparatus of claim 26, wherein said adhesion is provided by a temporary adhesive.

28. The apparatus of claim 24, wherein a further chamber is formed over the outer chamber and the protrusion thereof for compressing the protrusion for tending to reduce friction during the insertion of the sheath into the body cavity.

29. Apparatus for inserting an instrument in a body cavity, the apparatus including a sheath having a chamber for accommodating the instrument and a channel extending along the chamber, thereby defining an enclosed space; at least one hole in said chamber positioned within said enclosed space, means for temporarily applying fluid pressure to said enclosed space so as to cause the fluid to pass through the hole to build up pressure between the instrument and the chamber to facilitate removal of the instrument from the chamber.

30. Apparatus for inserting an instrument into a body cavity, the apparatus including a sheath for accommodating the instrument, wherein the sheath includes phosphorycholine.

31. The apparatus of claim 30, wherein the sheath is coated with phosphorycholine-containing polymers.

32. A medical instrument end cap having an aperture therethrough alignable with a working channel of the instrument, the end cap including a rim formation for abutting an edge region of said instrument and for preventing the passage of the instrument thought the aperture.

33. An assembly, including:
a medical instrument end cap having an aperture therethrough alignable with a working channel of the medical instrument, the end cap including a rim formation for abutting an edge region of the medical instrument and for preventing the passage of the medical instrument thought the aperture, and
a sheath attached to the end cap and for accommodating the medical instrument,
the sheath having at least one channel formed therein for allowing the passage of devices or fluid therealong.

34. An assembly, including:
a medical instrument having a working channel,
a medical instrument end cap having an aperture therethrough alignable with the working channel of the medical instrument, the end cap including a rim formation for abutting an edge region of the medical instrument and for preventing the passage of the medical instrument thought the aperture, and
a sheath attached to the end cap and for accommodating the medical instrument, the sheath having a first and second channel formed therein, and the medical instrument having a third channel formed therein,
one of said channels being for the passage of incoming irrigation fluid, one of said channels being for the passage of outgoing irrigation fluid and one of said channels being for performing a medical procedure.
